(19) United States
(12) Patent Application Publication
(10) Pub. No.: US 2026/0198906 A1
(43) Pub. Date: Jul. 16, 2026

(54) Pneumatic expansion-based soft nasal swab, sampling device, and sampling method

(71) Applicant: THE FIRST AFFILIATED HOSPITAL OF GUANGZHOU MEDICAL UNIVERSITY (GUANGZHOU RESPIRATORY CENTER), Guangzhou (CN)

(72) Inventors: Shaoqiang LI, Guangzhou (CN); Jingnan ZHAI, Guangzhou (CN); Weilong LI, Guangzhou (CN)

(21) Appl. No.: 19/361,515

(22) Filed: Oct. 17, 2025

Related U.S. Application Data
(63) Continuation of application No. PCT/CN2025/077615, filed on Feb. 17, 2025.

(30) Foreign Application Priority Data
Jan. 16, 2025 (CN) 202510069175.6

(51) Int. Cl. A61B 10/00

(52) U.S. Cl. CPC A61B 10/0045; A61B 2560/02

(57) ABSTRACT

A pneumatic expansion-based soft nasal swab includes a sampling segment configured to collect samples; a driving segment configured to axially provide a driving force for sampling; a support segment attached to the driving segment, which is configured to axially transmit the driving force to the sampling segment and provide support for the sampling segment; and a telescopic segment, with two ends respectively attached to the support segment and the sampling segment, which is configured to extend towards the sampling segment along the axial direction to insert the sampling segment into a sampling position in the nasal cavity of the subject, and configured to contract towards the support segment along the axial direction, when the sampling segment suffers from a resistance at the sampling position and applies a pressure force on the telescopic segment, and drive the sampling segment to rotate for automatic sampling.

### CROSS REFERENCE TO RELATED APPLICATIONS

The present application is a continuation of International Application No. PCT/CN2025/077615, filed on Feb. 17, 2025, which claims priority from Chinese Application No. 202510069175.6 filed on Jan. 16, 2025, all of which are hereby incorporated herein by reference.

### TECHNICAL FIELD

The present disclosure relates to the field of medical equipment, and in particular, to a pneumatic expansion-based soft nasal swab, a sampling device, and a sampling method.

### BACKGROUND

A nasal swab is a small tool used to collect substances from an inner wall of a nasal cavity for testing or experimentation. The existing nasal swab is mainly formed by a small wooden stick with a cotton tip at one end for sampling. During use, the smooth end of the small wooden stick is usually hold by a doctor to insert the cotton tip deep into the nostril and rotate the cotton tip to collect liquid or solid substances from the inner wall of the nasal cavity. The nasal swab with such structure generally has high rigidity and lacks elasticity, making it more prone to injuring the inner wall of the nasal cavity and causing significant discomfort to the subject to be sampled in the sampling process. In addition, contact with the subject is required during sampling, which would cause cross infection between the doctor and the subject to be sampled.

It is therefore desirable to improve the sampling experience for the subject to be sampled, such as through automatic sampling, eliminating cross infection caused by contact with the subject.

### SUMMARY

The present disclosure provides a pneumatic expansion-based soft nasal swab, a sampling device, and a sampling method, which is free from injury to the inner wall of the nasal cavity and unsatisfactory sampling experience due to inelasticity of the existing nasal swabs, without risk of cross infection caused by direct contact with the subject to be sampled through automatic sampling.

In a first aspect, a pneumatic expansion-based soft nasal swab is provided in the present disclosure, including:
a sampling segment, configured to collect samples in a nasal cavity of a subject to be sampled;
a driving segment, configured to axially provide a driving force for the sampling segment through air inflation;
a support segment attached to the driving segment, which is configured to transmit the driving force from the driving segment to the sampling segment in an axial direction, and provide support for the sampling segment during sampling; and
a telescopic segment, with two ends respectively attached to the support segment and the sampling segment, which is configured to extend towards the sampling segment along the axial direction under an action of the driving force to insert the sampling segment into a sampling position in the nasal cavity of the subject, and configured to contract towards the support segment along the axial direction, when the sampling segment suffers from a resistance at the sampling position and applies a pressure force on the telescopic segment, and drive the sampling segment to rotate for automatic sampling.

In a second aspect, an automatic sampling device is provided, including:
a nasal swab of cavity shaped; and
a pressurizing device for expanding the nasal swab and providing a driving force to the nasal swab for automatic sampling through air inflation,
wherein the nasal swab includes:
   a sampling segment, configured to collect samples in a nasal cavity of a subject to be sampled;
   a driving segment connected to the pressurizing device via a pipe, which can adjust a degree of the air inflation;
   a support segment attached to the driving segment, which is configured to transmit the driving force from the driving segment to the sampling segment in an axial direction, and provide support for the sampling segment during sampling; and
   a telescopic segment, with two ends respectively attached to the support segment and the sampling segment, which is configured to extend towards the sampling segment along the axial direction under an action of the driving force to insert the sampling segment into a sampling position in the nasal cavity of the subject, and configured to contract towards the support segment along the axial direction, when the sampling segment suffers from a resistance at the sampling position and applies a pressure force on the telescopic segment, and drive the sampling segment to rotate for automatic sampling.

In a third aspect, an automatic sampling method is further provided, including:
putting a nasal swab in place, and positioning the nasal swab, by a mechanical arm, close to a lower portion of a nasal cavity of a subject to be sampled;
activating a pressurizing device to expand the nasal swab and provide a driving force to the nasal swab, and extending the sampling segment into a sampling position of the nasal cavity under an action of the driving force;
applying a pressure force on the telescopic segment when the sampling segment suffers from resistance at the sampling position, and driving the telescopic segment to contract and rotate by the pressure force applied thereon;
driving the sampling segment attached to the telescopic segment to rotate for automatic sampling in the nasal cavity; and
placing, by the mechanical arm, the sampling segment in a collection box after completing sampling, deactivating the pressurizing device, and removing the nasal swab.

### BRIEF DESCRIPTION OF THE DRAWINGS

FIG. 1 is a perspective view of a soft nasal swab, in an expanded state, of an embodiment according to the present disclosure;

FIG. 2 is a perspective view of the soft nasal swab, in a contracted state, of the embodiment according to the present disclosure; and

FIG. 3 is a perspective view of an automatic sampling device according to an embodiment of the present disclosure.

### DETAILED DESCRIPTION OF THE EMBODIMENTS

The accompanying drawings of the present disclosure are only intended for illustrative purposes only and should not be construed as limiting the present disclosure. In order to better illustrate the following embodiments, some components in the accompanying drawings may be omitted, enlarged or reduced, and do not represent actual product dimensions. It will be understood by those skilled in the art that certain well-known structures and their descriptions in the accompanying drawings may be omitted.

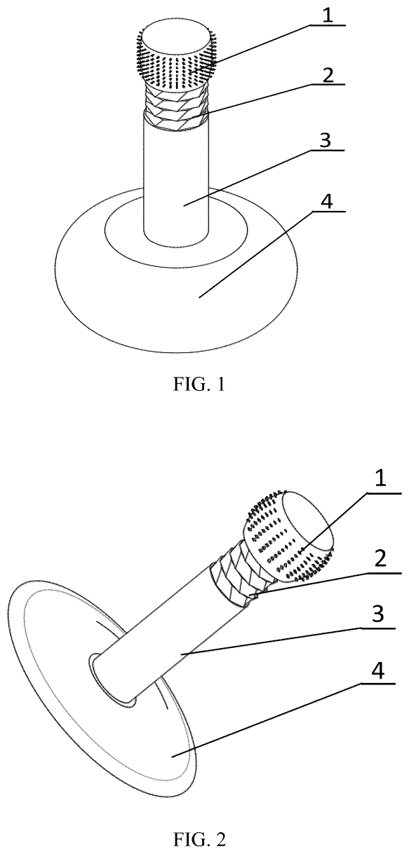

FIG. 1 and FIG. 2 provide a pneumatic expansion-based soft nasal swab, including a sampling segment 1, telescopic segment 2, an auxiliary support segment 3, and an axial driving segment 4, which are sequentially connected with each other.

The nasal swab in the present embodiment is of cavity-shaped. The sampling segment 1 is configured to collect samples in a nasal cavity of a subject to be sampled. The driving segment 4 is configured to axially provide a driving force for the sampling segment through air inflation, which can control degrees of the air inflation in the cavity. The support segment 3 is attached to the driving segment 4, which is configured to transmit the driving force from the driving segment 4 to the sampling segment 1 in an axial direction, and provide support for the sampling segment 1 during sampling. Two ends of the telescopic segment 2 are respectively attached to the support segment 3 and the sampling segment 1. The telescopic segment 2 in the present embodiment is configured to extend towards the sampling segment 1 along the axial direction under an action of the driving force to insert the sampling segment 1 into a sampling position in the nasal cavity of the subject. The telescopic segment 2 in the present embodiment is further configured to contract towards the support segment 3 along the axial direction, when the sampling segment 1 suffers from a resistance at the sampling position and applies a pressure force on the telescopic segment 2, and drive the sampling segment 1 to rotate for automatic sampling during contraction.

In this embodiment, the axial driving segment 4 provides pneumatic power supply for the nasal swab, which enables different segments of the nasal swab to deform in a way that the nasal swab moves within the nasal cavity in a sampling direction. The axial driving segment 4 also provides buffering for the air inflation of the nasal swab, so as to control degrees of the air inflation in the cavity. The auxiliary support segment 3 provides a support force for the telescopic segment 2 and controls transmission of the driving force during use. The telescopic segment 2 provides changes in axial and radial forces for sampling, thereby driving the sampling segment 1 to achieve rotational and telescopic sampling. In such way, the nasal swab achieves automatic sampling to avoide injury to the nasal cavity, thereby improving subject's experience. The sampling segment 1 is in direct contact with the nasal cavity of the subject, which has increased contact area for sampling due to the brushes design. The sampling segment 1 is connected to the telescopic segment 2 so that the brushes can achieve elastically extension, contraction and rotation in the sampling process, thereby achieving automatic sampling in large area, and thus improving sampling experience and sampling accuracy without direct contact with the subject to be sampled.

Particularly, the sampling segment 1 in the present embodiment is a spherical cavity having two flat ends, with a plurality of brushes uniformly distributed on the lateral side. One end of the spherical cavity is connected with the telescopic segment 2, and the other end is a smooth surface.

In this embodiment, no brushes are arranged on the smooth surface, so that the subject will not feel prickling sensation when the smooth surface suffers resistance due to contact with the nasal cavity. Furthermore, the smooth surface can fully and axially transmit the pressure force applied by the sampling segment 1 due to resistance, avoiding dispersion of force. The plurality of brushes uniformly distributed on the lateral side of the sampling segment 1 significantly increase the sampling coverage area, therefore when the sampling segment 1 is driven to rotate, the large-area brushes may collect more samples at the same time, which ensures concentration of the collected sample, thereby further improving sampling accuracy.

The telescopic segment 2 in the present embodiment is a telescope cavity, which can be extended and contracted. The telescopic segment 2 formed by a plurality of annular layers with the diameter sequentially decreasing in the axial direction from the auxiliary support segment 3 to the sampling segment 1. When the telescopic segment 2 is pressed by the sampling segment 1, the plurality of annular layers are contracted axially from the sampling segment 1 to the auxiliary support segment 3. In such configuration, the auxiliary support segment 3 achieves elastic sampling of the nasal swab by coupling the rotation sampling and the front-end pressure together, thus improving the subject's sampling experience.

In this embodiment, the telescopic segment 2 particularly includes four annular layers. When the sampling segment 1 encounters resistance in the nasal cavity, the smooth surface of the telescopic segment 2 axially applies the pressure force to the telescopic segment 2. Due to the smaller diameter at the top and large diameter at the bottom of the telescopic segment 2, the top layer close to the sampling segment 1 is contracted toward to the auxiliary support segment 3 under the pressure force, until all the layers are contracted.

More preferably, each annular layer is formed by a plurality of diamond surfaces which are connected with each other in a circumferential direction. With such configuration, when the telescopic segment 2 is subjected to pressure force applied by the sampling segment 1 due to resistance from the nasal cavity, the diamond surfaces of each annular layer convert at least portion of the axial pressure force into a tangential force, causing the plurality of annular layers to rotate in the circumferential direction.

In this embodiment, the oblique edges of the plurality of diamond surfaces of each annular layer are connected end to end, and the upper and lower parallel edges of the diamond surfaces form the edges of the annular layer, which keeps the plurality of annular layers coplanar. Accordingly, when the telescopic segment 2 is subjected to axial pressure, part of the force undergoes unstable deformation due to the special structure of the diamond surfaces, resulting in that portion of the axial force is converted into the tangential force, which drives the diamond surfaces to rotate.

The auxiliary support segment 3 in the present embodiment is formed in a cylindrical cavity, with one end connected to the telescopic segment 2 and the other end connected to the axial driving segment 4. The auxiliary support segment 3 has an axial length of H3, the telescopic segment 2 has an axial length of H2, and the sampling segment 1 has an axial length of H1, where H3>H1+H2.

In this embodiment, the cylindrical cavity of the auxiliary support segment 3 consistently maintains a rigid cylindrical shape, which can always maintain high-strength support force regardless of the inflation state or force condition of the telescopic segment 2 and the sampling segment 1, thereby controlling the movement in the sampling direction and restricting the axial transmission of the force when resistance is encountered during sampling. Greater axial length of the auxiliary support segment 3 generally provides stronger support force. Particularly, in the present embodiment the axial length of the auxiliary support segment may be greater than the total axial length of the telescopic segment and the sampling segment, so that the auxiliary support segment can provide enough support force axially to confront the pressure force axially transmitted downward by the telescopic segment, thereby ensuring rotation of the sampling segment.

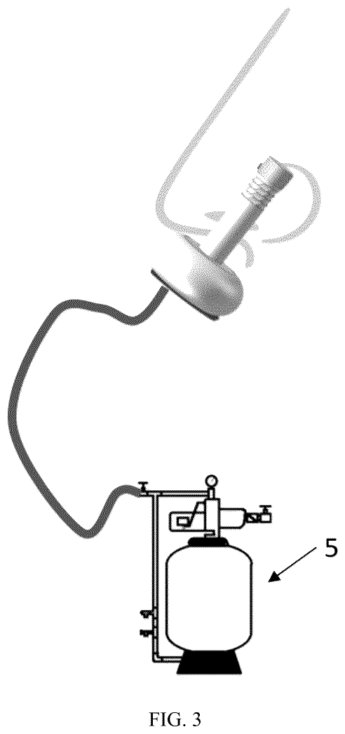

The axial driving segment 4 in the present embodiment is a drum-shaped cavity including a top surface and a bottom surface which have the same diameter. The bottom surface may be connected to an external pressurizing device 5 via a pipe, see FIG. 3, and the axial driving segment 4 expands axially when the pressurizing device 5 is activated and pushes the sampling segment 1 to move forward.

In this embodiment, the top surface and the bottom surface of the axial driving segment 4 have the same diameter, so that when the nasal swab is in a contracted state without inflation, the rigid top surface and the rigid auxiliary support segment 3 support each other, allowing rapid deformation in place during the next inflation for sampling. The bottom surface quickly directs, through the pipe, gas generated by the pressurizing device along the fixed auxiliary support segment 3 into the sampling segment 1, thereby achieving rapid sampling of the sampling segment 1.

The top surface has a diameter of D4, the auxiliary support segment has a diameter of D3, the bottom of the telescopic segment has a diameter of D22, the top of the telescopic segment has a diameter of D21, the diameter of the brush formed at the outer edge of the sampling segment, namely the maximum diameter of the sampling segment is D1, and the pipe has a diameter of D5, where D4>D1>D3=D22>D21>D5.

With different diameters of each segment of the nasal swab, each segment of the soft nasal swab can perform different actions, making it adapt to the internal environment of the nasal cavity for deformation. In particular, the pipe with small diameter can increase the airflow speed during inflation. Gradually decreasing diameter of the telescopic segment from the bottom to the top can achieve axial telescoping. With the diameter of the outer edge of the brushes greater than that of the telescopic segment, the contact area for sampling can be increased. In addition, the equal diameters of the auxiliary support segment and the bottom of the telescopic segment can improve efficiency of transmission of the axial force.

In this embodiment, with the diameter of the pipe much smaller than that of the drum surface of the axial driving segment 4, the airflow speed during inflation thus is increased, which can achieve rapid inflation. The diameter of the auxiliary support segment 3 is approximately half of that of the drum surface of the axial driving segment 4, which can not only meet the diameter requirement for insertion into the nasal cavity, but also provide sufficient support force for the telescopic segment 2 and the axial driving segment 4. The diameter of the top of the telescopic segment 2 is slightly smaller than that of the bottom of the telescopic segment 2, and the difference between the two diameters is preferably the total wall thicknesses of three annular layers. The diameter of the outer edge of the brushes is greater than the telescopic segment 2 and the auxiliary support segment 3, ensuring that the brushes can be in full contact with the inner wall of the nasal cavity after insertion into the nasal cavity, thereby increasing the concentration of the collected sample.

A sampling method conducted by a sampling device including the pneumatic expansion-based soft nasal swab mentioned above is further provided.

The nasal swab is firstly positioned close to the lower portion of the nostril of the subject through a mechanical arm.

The pressurizing device 5 is activated to inflate the soft nasal swab, so that the axial driving segment 4, the auxiliary support segment 3, the telescopic segment 2, and the sampling segment 1 of the nasal swab are sequentially expanded to extend the sampling segment 1 into the nasal cavity.

The telescopic segment 2 is contracted and rotated in a circumferential direction under pressure force applied by the sampling segment 1 due to resistance at the sampling position inside the nasal cavity.

The sampling segment 1 connected with the telescopic segment 2 is driven to rotate to complete automatic sampling in the nasal cavity.

Finally, the sampling segment 1 is placed in a collection box through the mechanical arm after completing sampling by the nasal swab. The pressurizing device 5 is deactivated and the soft nasal swab is removed to prepare for a next sampling.

With such sampling method, the contact with the subject to be sampled is eliminated in the sampling process, thereby reducing cross infection. The sampling method is easy to operate in a way that different actions at different stages are achieved using one driving source by gas pressure driving and the special structural design of different parts of the nasal swab. In addition, the soft material of the nasal swab reduces the safety risk and its deformation is adapted to the change in the internal environment of the nasal cavity, which enables better coverage effect of the sampling area.

Specifically, in the present sampling method, the air quickly flows into the sampling segment 1 through the cylindrical cavity after the driving segment 4 is inflated and expanded, and the sampling segment 1 is pushed into the nasal cavity;

The axial driving segment 4 and the auxiliary support segment 3 provide a support force for the telescopic segment 2, especially all annular layers of the telescopic segment 2 are fully expanded after continuous inflation.

With expansion of the annular layers of the telescopic segment 2, the sampling segment 1 is gradually extended into the nasal cavity with resistance gradually increasing. The sampling segment 1 applies a pressure force to the telescopic segment 2 when the sampling segment 1 reaches the sampling position inside the nasal cavity at which the resistance on the sampling segment 1 reaches the maximum.

According to the present embodiment, elastic sampling of the nasal swab is achieved through specific action changes of the driving segment 4, the auxiliary support segment 3, and sampling segment 1 during inflation.

Specifically, the pressure force is axially transmitted to the top of the telescopic segment 2 by the top of the sampling segment 1 which is a smooth and flat surface, when the sampling segment 1 reaches the sampling position inside the nasal cavity at which the resistance reaches the maximum.

Then, a part of the pressure force is axially transmitted from the top of the telescopic segment 2 to the bottom of the telescopic segment 2 through the plurality of annular layers, which causes the plurality of annular layers to axially contract under the action of this part of pressure force.

While the other part of the pressure force is transmitted tangentially along the diamond surfaces of each layer by the top of the telescopic segment 2. As the auxiliary support segment 3 connected with the telescopic segment 2 provides a support force for the bottom of the telescopic segment 2 to prevent such part of pressure force from being further axially transmitted, the telescopic segment 2 is driven to rotate tangentially in the circumferential direction under the action of such pressure force.

According to the present embodiment, a large-area rotational sampling is achieved through the specific action changes in the force state of the telescopic segment at various stages.

Apparently, the above-mentioned embodiments of the present disclosure are only examples for clearly explaining the technical schemes of the present disclosure, and are not intended to limit the specific implementations of the present disclosure. Any modifications, equivalent substitutions or improvements made within the spirit and principle of the claims of present disclosure shall be included within the protection scope of the claims of the present disclosure.

What is claimed is:

1. A pneumatic expansion-based soft nasal swab, comprising:
a sampling segment, configured to collect samples in a nasal cavity of a subject to be sampled;
a driving segment, configured to axially provide a driving force for the sampling segment through air inflation;
a support segment attached to the driving segment, which is configured to transmit the driving force from the driving segment to the sampling segment in an axial direction, and provide support for the sampling segment during sampling; and
a telescopic segment, with two ends respectively attached to the support segment and the sampling segment, which is configured to extend towards the sampling segment along the axial direction under an action of the driving force to insert the sampling segment into a sampling position in the nasal cavity of the subject, and configured to contract towards the support segment along the axial direction, when the sampling segment suffers from a resistance at the sampling position and applies a pressure force on the telescopic segment, and drive the sampling segment to rotate for automatic sampling.

2. The pneumatic expansion-based soft nasal swab according to claim 1, wherein the sampling segment is in form of a spherical cavity having two flat ends, with a plurality of brushes uniformly distributed on a lateral side of the spherical cavity, and the telescopic segment being attached to an underneath of the spherical cavity.

3. The pneumatic expansion-based soft nasal swab according to claim 1, wherein the telescopic segment is in form of a telescopic cavity, comprising a plurality of annular layers, with a diameter axially decreases from the support segment to the sampling segment, and the plurality of annular layers are sequentially contracted from the sampling segment to the support segment along the axial direction when pressed by sampling segment due to resistance at the sampling position.

4. The pneumatic expansion-based soft nasal swab according to claim 3, wherein each annular layer is formed by a plurality of diamond surfaces which are connected with each other in a circumferential direction, which is configured to convert at least a portion of the axial pressure force into a tangential force, so as to drive the sampling segment to rotate in the circumferential direction during contraction.

5. The pneumatic expansion-based soft nasal swab according to claim 1, wherein the support segment is formed in a cylindrical cavity, with a top end attached to an underneath of the telescopic segment and a bottom end attached to the driving segment, and an axial length of the support segment is greater than the total axial length of the telescopic segment and the sampling segment.

6. The pneumatic expansion-based soft nasal swab according to claim 1, wherein the axial driving segment is in form of a drum-shaped cavity, which has a top surface and a bottom surface with same diameter.

7. The pneumatic expansion-based soft nasal swab according to claim 6, wherein the top surface of the axial driving segment has a diameter of D4, the auxiliary support segment has a diameter of D3, a bottom portion of the telescopic segment has a diameter of D22, a top portion of the telescopic segment has a diameter of D21, and a maximum diameter of the sampling segment is D1, which satisfies D4>D1>D3=D22>D21.

8. An automatic sampling device, comprises:
a nasal swab of cavity shaped; and
a pressurizing device for expanding the nasal swab and providing a driving force to the nasal swab for automatic sampling through air inflation,
wherein the nasal swab comprises:
a sampling segment, configured to collect samples in a nasal cavity of a subject to be sampled;
a driving segment connected to the pressurizing device via a pipe, which can adjust a degree of the air inflation;
a support segment attached to the driving segment, which is configured to transmit the driving force from the driving segment to the sampling segment in an axial direction, and provide support for the sampling segment during sampling; and
a telescopic segment, with two ends respectively attached to the support segment and the sampling segment, which is configured to extend towards the sampling segment along the axial direction under an action of the driving force to insert the sampling segment into a sampling position in the nasal cavity of the subject, and configured to contract towards the support segment along the axial direction, when the sampling segment suffers from a resistance at the sampling position and applies a pressure force on the telescopic segment, and drive the sampling segment to rotate for automatic sampling.

9. The automatic sampling device according to claim 8, wherein the sampling segment is in form of a spherical cavity having two flat ends, with a plurality of brushes uniformly distributed on a lateral side of the spherical cavity, and the telescopic segment being attached to an underneath of the spherical cavity.

10. The automatic sampling device according to claim 8, wherein the telescopic segment is in form of a telescopic cavity, comprising a plurality of annular layers, with a diameter axially decreases from the sampling segment to the support segment, and the plurality of annular layers are sequentially contracted from the sampling segment to the support segment along the axial direction when pressed by sampling segment due to resistance at the sampling position.

11. The automatic sampling device according to claim 10, wherein each annular layer is formed by a plurality of diamond surfaces which are connected with each other in a circumferential direction, which is configured to convert at least a portion of the axial pressure force into a tangential force, so as to drive the sampling segment to rotate in the circumferential direction during contraction.

12. The automatic sampling device according to claim 8, wherein the support segment is formed in a cylindrical cavity, with a top end attached to an underneath of the telescopic segment and a bottom end attached to the driving segment, and an axial length of the support segment is greater than the total axial length of the telescopic segment and the sampling segment.

13. The automatic sampling device according to claim 8, wherein the axial driving segment is in form of a drum-shaped cavity, which has a top surface and a bottom surface with same diameter, the bottom surface being connected to the pressurizing device via the pipe.

14. The automatic sampling device according to claim 8, wherein the top surface has a diameter of D4, the auxiliary support segment has a diameter of D3, a bottom portion of the telescopic segment has a diameter of D22, a top portion of the telescopic segment has a diameter of D21, the sampling segment has a maximum diameter of D1, and the pipe has a diameter of D5, which satisfies D4>D1>D3=D22>D21>D5.

15. A sampling method conducted by the sampling device according to claim 8, comprising steps of:
putting a nasal swab in place, and positioning the nasal swab, by a mechanical arm, close to a lower portion of a nasal cavity of a subject to be sampled;
activating a pressurizing device to expand the nasal swab and provide a driving force to the nasal swab, and extending the sampling segment into a sampling position of the nasal cavity under an action of the driving force;
applying a pressure force on the telescopic segment when the sampling segment suffers from resistance at the sampling position, and driving the telescopic segment to contract and rotate by the pressure force applied thereon;
driving the sampling segment attached to the telescopic segment to rotate for automatic sampling in the nasal cavity; and
placing, by the mechanical arm, the sampling segment in a collection box after completing sampling, deactivating the pressurizing device, and removing the nasal swab.

16. The sampling method according to claim 15, wherein the telescopic segment is in form of a telescopic cavity, comprising a plurality of annular layers, each annular layer being formed by a plurality of diamond surfaces which are connected with each other in a circumferential direction, and wherein during expansion, the diamond surfaces are extended towards the sampling segment along the axial direction to insert the sampling segment into the sampling position.

17. The sampling method according to claim 16, wherein a portion of the pressure force is axially transmitted to a bottom portion of the telescopic segment, and the diamond surfaces of the telescopic segment are contracted under the pressure force axially transmitted; another portion of the pressure force is tangentially transmitted in a circumferential direction via the diamond surfaces, and the telescopic segment is rotated in the circumferential direction under the pressure force tangentially transmitted.
